(19) United States
(12) Patent Application Publication
(10) Pub. No.: US 2016/0287367 A1
(43) Pub. Date: Oct. 6, 2016

(54) BIORESORBABLE SEPTOPLASTY CLOSURE IMPLANT

(71) Applicant: Daniel A. Rontal, Birmingham, MI (US)

(72) Inventor: Daniel A. Rontal, Birmingham, MI (US)

(21) Appl. No.: 15/085,250

(22) Filed: Mar. 30, 2016

Related U.S. Application Data
(60) Provisional application No. 62/140,146, filed on Mar. 30, 2015.

(51) Int. Cl. A61F 2/00; A61F 2/18

(52) U.S. Cl. CPC A61F 2/0063; A61F 2/186; A61F 2/0059; A61F 2002/0068; A61F 2210/0004; A61F 2250/0067

(57) ABSTRACT

A bioresorbable implant and associated method for use in conjunction with septoplasty procedure comprises a thin, at least partially flexible mesh sheet constructed with struts of material intersecting at points defining openings entirely through the mesh sheet, and wherein the sheet defines a plane with opposing side surfaces. A plurality of projections extend away from the sheet on both side surfaces thereof. The openings through the mesh sheet have minimum dimensions in the range of 2 to 8 mm, and the projections have a length in the range of 1 to 5 mm. The openings in the mesh sheet may define regular polygons, whereby the struts may have minimum dimensions in the range of 2 to 8 mm. The projections may extend from the intersections of the struts, directly from the struts, or both from the intersections and the struts. A method of performing septoplasty in accordance with the invention is also disclosed.

### REFERENCE TO RELATED APPLICATION

This application claims priority to U.S. Provisional Patent Application Ser. No. 62/140,146, filed Mar. 30, 2015, the entire content of which is incorporated herein by reference.

### FIELD OF THE INVENTION

This invention relates generally to septoplasty and, in particular, to a bioresorbable implant for reapproximating the muco-perichondrial flaps in conjunction with such procedures.

### BACKGROUND OF THE INVENTION

Septoplasty is one of the most commonly performed ENT surgeries. It involves straightening or removing the twisted portions of the nasal septum. The septum, which is composed of cartilage anteriorly and bone posteriorly, can become twisted through trauma or can simply grow that way. A deviated septum can lead to nasal obstruction and obstruct the outflow of the sinuses.

Septoplasty is typically performed with all of the incisions placed inside of the nose. The mucous membrane of the septum is lifted off of the cartilage and bone by dissecting below the nutrient layer of these tissues. At the completion of the procedure, the two sides of the septum's mucous membrane, or flaps, need to be reapproximated and the dead space between them closed down. This ensures the septum is straight and that a hematoma does not develop between the flaps. Hematomas can lead to loss of the residual cartilage which can destabilize the nose causing collapse.

Re-approximation can be performed in several different fashions. Most surgeons suture the two sides together by passing the needle of the dissolvable suture through from one side to the other. This process is technically challenging, leads to incomplete re-approximation where it is difficult to suture-specifically posteriorly and superiorly, and frequently causes lacerations in the lateral nasal wall as the needle is brought through the nose.

### SUMMARY OF THE INVENTION

This invention resides in a bioresorbable implant and associated method for use in conjunction with a septoplasty procedure. The implant comprises a thin, at least partially flexible mesh sheet constructed with struts of material intersecting at points defining openings entirely through the mesh sheet, and wherein the sheet defines a plane with opposing side surfaces. A plurality of projections connected to the mesh sheet extend away from the sheet on both side surfaces thereof. The openings through the mesh sheet have minimum dimensions in the range of 2 to 8 mm, and the projections have a length in the range of 1 to 5 mm.

The implant may be composed of a mixture of poly lactic acid (PLA) and poly lactic-co-glycolic acid (PLGA) or poly glycolic acid (PGA) and PGLA, and the constituent ingredients may be formulated to support biodegradability on the order of a week, more or less. The openings in the mesh sheet may define regular polygons, whereby the struts may have minimum dimensions in the range of 2 to 8 mm. The projections may extend from the intersections of the struts, directly from the struts, or both from the intersections and the struts.

The projections may be mirror images of one another on the two side surfaces of the mesh material, and may be substantially perpendicular to the plane of the mesh material. The projections may terminate in ends with prongs, bristles, barbs or hooks, and the implant may further including a local anesthetic.

A method of performing septoplasty, in accordance with the invention includes the step of making an incision and removing unwanted cartilage or bone from the septum, thereby creating a void between opposing muco-perichondrial flaps. A bioresorbable implant constructed in accordance with the invention is inserted into the void, and the muco-perichondrial flaps are pressed against the implant such that the projections of the implant impale the muco-perichondrial flaps and hold the flaps against one another for a desired period of time such a one week. The projects may or may not penetrate entirely through the muco-perichondrial flaps.

### BRIEF DESCRIPTION OF THE DRAWINGS

FIG. 1 is a drawing of an embodiment of the invention based on a hexagonal mesh;

FIG. 2 illustrates a square mesh;

FIG. 3 shows a triangular mesh with indications regarding the way in which barbs or prongs may attach;

FIG. 4 depicts various alternative barb shapes;

FIG. 5A is a simplified drawing of a deviated septum;

FIG. 5B shows an incision made as part of a septoplasty procedure;

FIG. 5C illustrates a mesh according to the invention inserted into the site where cartilage and/bone were removed; and

FIG. 5D shows the muco-perichondrial flaps pressed against the implant such that the bristles impale the flaps to re-approximating them.

### DETAILED DESCRIPTION OF THE INVENTION

This invention improves upon septoplasty procedures by providing a bio-resorbable implant used to coat the two muco-perichondrial flaps in a more efficient manner, thereby minimizing the risk of dead space or nasal side-wall lacerations. To achieve bio-resorbability, the implant may be composed of a mixture of poly lactic acid (PLA) and poly lactic-co-glycolic acid (PLGA) or poly glycolic acid (PGA) and PGLA. Those of skill in the art will appreciate that a combination of such materials may be used to achieve a structural integrity lasting a desired length of time. In the preferred embodiments of this invention the implant which will dissolve in approximately one week.

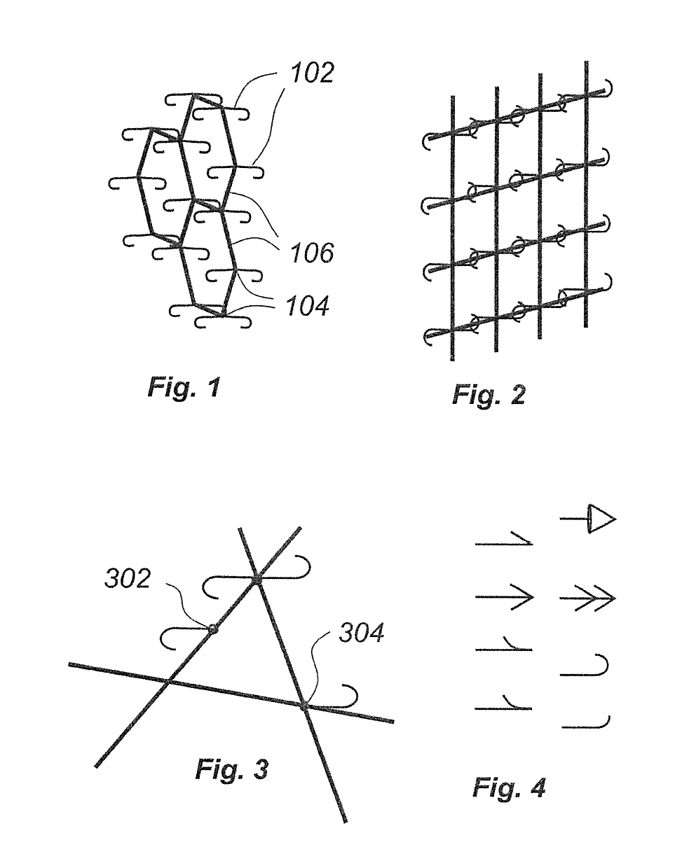

The implant itself comprises a mesh that is at least somewhat flexible and sufficiently thin that the so that the two sides of the muco-perichondrial flaps will be able to touch internally between the mesh. FIG. 1 is a drawing of an embodiment of the invention based on a hexagonal mesh. As shown in FIGS. 2, 3, other mesh patterns may be used including square, and triangular. Indeed, any other repeating (or even non-repeating) pattern may be used, including overlapping circles and ovals, etc., so long as the limitations herein regarding composition, construction and size are met.

In the preferred embodiments, regardless of the mesh pattern, the minimum dimension of the openings though the mesh are on the order of 2-8 mm, more preferably in the range of 4-6 mm, to allow for fusion of the flap surfaces. If a repeating geometric pattern is used the struts that make up the pattern have lengths in the range of 2-8 mm, more preferably in the range of 4-6 mm.

The mesh sheet defines a plane with opposing side surfaces. In accordance with the invention, a plurality of prongs, bristles, barbs or hooks (herein after “projections”) extend from both side surfaces of the mesh. The projections are preferably 1-5 mm in length, more preferably 1-3 mm. In the preferred embodiments, these projections protrude from the intersections 104 of the struts 106 that comprise the mesh, and may be mirror images of one another on the two sides. However, as shown in FIG. 3, the projections on the two sides may not be symmetrical, and may extend from points 302 along the struts and/or the intersections (at 304).

The projections preferably but not necessarily extend perpendicularly from the plane of the mesh proper, though this is not a requirement. For example, in one embodiment, the projections may comprise a bed of straight, curved or bent structures extending at the same or different angles from the plane of the mesh. FIG. 4 illustrates several alternative projection designs, which may include two- and three-dimensional pointed structures and hooks or barbs that repeat along the shaft of the projection.

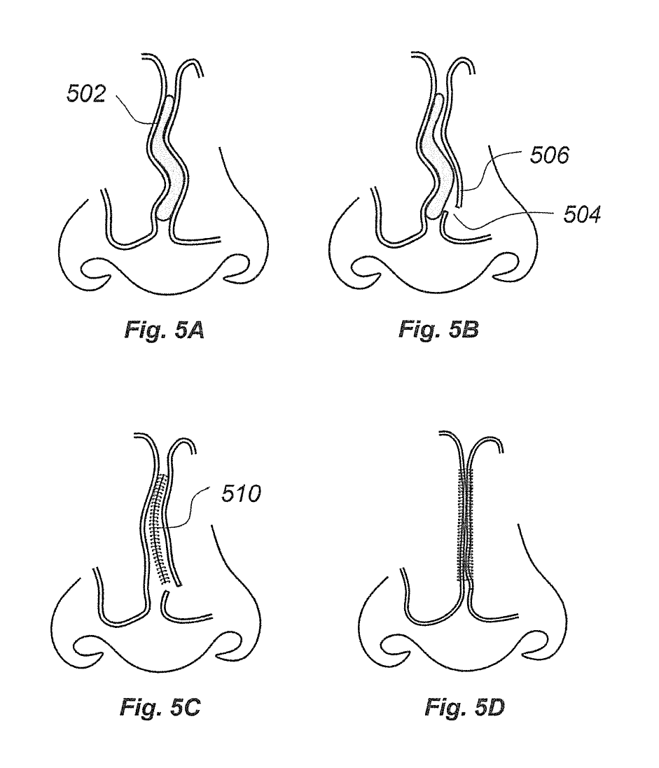

FIG. 5A is a simplified drawing of a deviated septum including bone and/or cartilage 502 to be removed. FIG. 5B illustrates an anterior incision 504 (hemi-transfixion or Killian) enabling the mucous membrane 506 of the septum is lifted off of the cartilage and bone by dissecting below the nutrient layer of these tissues. FIG. 5C illustrates a mesh according to the invention 510 inserted into the site where cartilage and/bone were removed. In practice, the surgeon will estimate the size of the mesh sheet needed and cut the material to size with scissors.

FIG. 5D shows the muco-perichondrial flaps pressed against the implant such that the bristles impale the flaps to re-approximating them. Note that in preferred embodiments the projections extend entirely through the flaps though this is not strictly necessary so long as the structure of the projections holds the flaps together for sufficiently long to promote fusion. The anterior incision can then be closed in the usual manner or not at all if the implant is anterior enough. The use of this implant with greatly reduce the surgical time and make the closure of the flaps more precise.

The bio-resorbable mesh material can be impregnated with different medications as well. One potential possibility would be local anesthetics. By combining the local anesthetic directly into the material, post-operative pain would be greatly reduced, possibly removing the necessity for taking postoperative pain medications at all.

1. An implant for use in conjunction with septoplasty, comprising:
a thin, at least partially flexible mesh sheet constructed with struts of material intersecting at points defining openings entirely through the mesh sheet;
the mesh sheet defining a plane with opposing side surfaces;
a plurality of projections connected to the mesh sheet and extending away from the sheet on both side surfaces thereof;
wherein the implant is composed of a bioresorbable material;
wherein the openings through the mesh sheet have minimum dimensions in the range of 2 to 8 mm; and
wherein the projections have a length in the range of 1 to 5 mm.

2. The implant of claim 1, wherein the implant is composed of a mixture of poly lactic acid (PLA) and poly lactic-co-glycolic acid (PLGA) or poly glycolic acid (PGA) and PGLA.

3. The implant of claim 1, wherein:
the openings in the mesh sheet define regular polygons; and
the struts defining the polygons have minimum dimensions in the range of 2 to 8 mm.

4. The implant of claim 1, wherein the projections extend from the intersections.

5. The implant of claim 1, wherein the projections extend from the struts.

6. The implant of claim 1, wherein the projections are mirror images of one another on the two side surfaces of the mesh material.

7. The implant of claim 1, wherein the projections are substantially perpendicular to the plane of the mesh material.

8. The implant of claim 1, wherein the projections terminate in ends with prongs, bristles, barbs or hooks.

9. The implant of claim 1, further including a local anesthetic.

10. A method of performing septoplasty, comprising the steps of:
making an incision and removing unwanted cartilage or bone from the septum, thereby creating a void between opposing muco-perichondrial flaps;
providing the implant of claim 1 and inserting the implant into the void; and
pressing the muco-perichondrial flaps against the implant such that the projections of the implant impale the muco-perichondrial flaps and hold the flaps against one another for a desired period of time.

11. The method of claim 10, wherein the desired period of time is approximately one week.

12. The method of claim 10, wherein at least some of the projections penetrate entirely through one or both of the muco-perichondrial flaps.
